(19) United States
(12) Patent Application Publication
(10) Pub. No.: US 2026/0060752 A1
(43) Pub. Date: Mar. 5, 2026

(54) AUTOMATED METHOD FOR INFERIOR ALVEOLAR NERVE CANAL SEGMENTATION OF THREE DIMENSIONAL VOLUME DATA AND COMPUTER READABLE MEDIUM HAVING PROGRAM FOR PERFORMING THE METHOD

(71) Applicant: IMAGOWORKS INC., Seoul (KR)

(72) Inventors: Yunseung HYUN, Seoul (KR); Youngjin OH, Seoul (KR); Sojeong CHEON, Seoul (KR); Hannah KIM, Seoul (KR); Dongwook LEE, Seoul (KR)

(73) Assignee: IMAGOWORKS INC., Seoul (KR)

(21) Appl. No.: 19/310,357

(22) Filed: Aug. 26, 2025

(30) Foreign Application Priority Data
Aug. 28, 2024 (KR) 10-2024-0115978
Oct. 14, 2024 (WO) PCT/KR2024/015513

(51) Int. Cl. A61B 34/10; G06T 7/11

(52) U.S. Cl. CPC A61B 34/10; G06T 7/11; A61B 2034/105; A61B 2034/108; G06T 2207/20084; G06T 2207/30036

(57) ABSTRACT

An automated method for an inferior alveolar nerve canal segmentation of three dimensional volume data according to the present inventive concept, the method includes determining a central feature point and an end feature point from the three dimensional volume data including an inferior alveolar nerve canal, separating a central region of the inferior alveolar nerve canal from the three dimensional volume data based on the central feature point, separating an end region of the inferior alveolar nerve canal from the three dimensional volume data based on the end feature point, and reconstructing the inferior alveolar nerve canal based on the central region of the inferior alveolar nerve canal and the end region of the inferior alveolar nerve canal.

### PRIORITY STATEMENT

This application claims priority under 35 U.S.C. § 119 to Korean Patent Application No. 10-2024-0115978, filed on Aug. 28, 2024 in the Korean Intellectual Property Office (KIPO) and International Patent Application No. PCT/KR2024/015513 filed on Oct. 14, 2024, the contents of which are herein incorporated by reference in their entireties.

### BACKGROUND

### 1. Technical Field

Embodiments relate to an automated method for an inferior alveolar nerve canal segmentation of three dimensional volume data and computer readable medium having program for performing the method. More particularly, embodiments relate to an automated method for an inferior alveolar nerve canal segmentation of three dimensional volume data and computer readable medium having program for performing the method for automatically performing a deep learning to reduce a time and an effort required to separate the inferior alveolar nerve canal from the three volume data.

### 2. Description of the Related Art

Three dimensional volume data represents accumulated data of a three dimensional image (e.g., a voxel) or a second dimensional image, such as those from CT (Computed Tomography), CBCT (Cone-Beam CT), or MRI (Magnetic Resonance Imaging). In dentistry and plastic surgery, the three dimensional volume data is limited to a head and neck region and used to diagnose and treat a patient' maxillofacial and oral regions.

An inferior alveolar nerve canal, located within a mandible, is a large nerve branch responsible for a sensation. When the inferior alveolar nerve canal is damaged, an aftereffect such as a synesthesia may occur. Therefore, a process of three-dimensionally analyzing the inferior alveolar nerve canal is required to establish a diagnosis, a treatment, and a surgical plan for a dental treatment such as an wisdom tooth extraction, a dental implant placement, and an orthognathic surgery, or to compare before and after surgery.

However, since the three dimensional volume data only has an intensity information of a single channel, in order to three-dimensionally analyzing specific region, a process of segmenting the specific region or reconstructing segmented data into a three dimensional model is required. Since the inferior alveolar nerve canal exists inside the mandible, a location of the inferior alveolar nerve canal should be estimated through a cutting plane of the mandible. In addition, a metal material such as a maxillofacial surgical plate, a metal dental prostheses, and an orthodontic device may cause a metal artifact in three dimensional volume data (e.g., CT or CBCT) using X-ray. Therefore, it may be difficult to accurately separate the inferior alveolar nerve canal.

### SUMMARY

Embodiments provide a method of representing an automated method for an inferior alveolar nerve canal segmentation of three dimensional volume data for automatically performing a deep learning to separate an inferior alveolar nerve canal from the three dimensional volume data and to improve an accuracy.

Embodiments provide a computer readable medium having program for executing the automated method for the inferior alveolar nerve canal segmentation of the three dimensional volume data.

In an example automated method for an inferior alveolar nerve canal segmentation of three dimensional volume data according to the present inventive concept, the method includes determining a central feature point and an end feature point from the three dimensional volume data including an inferior alveolar nerve canal, separating a central region of the inferior alveolar nerve canal from the three dimensional volume data based on the central feature point, separating an end region of the inferior alveolar nerve canal from the three dimensional volume data based on the end feature point, and reconstructing the inferior alveolar nerve canal based on the central region of the inferior alveolar nerve canal and the end region of the inferior alveolar nerve canal.

In an embodiment, the three dimensional volume data may be input into a first artificial intelligence neural network, and the central feature point and the end feature point may be determined from the first artificial intelligence neural network.

In an embodiment, the central feature point may include at least three feature points.

In an embodiment, the end feature point may include at least two feature points.

In an embodiment, the separating the central region of the inferior alveolar nerve canal may include generating a virtual curve based on the central feature point, generating a cutting plane of the central region of the inferior alveolar nerve canal based on the virtual curve, and separating the central region of the inferior alveolar nerve canal based on the cutting plane of the central region of the inferior alveolar nerve canal to generate central separation data.

In an embodiment, the virtual curve may be a spline curve which directly passes through the central feature point.

In an embodiment, the virtual curve may be a Bezier curve which does not directly pass through the central feature point.

In an embodiment, the cutting plane may be perpendicular to the virtual curve.

In an embodiment, an intermediate cutting plane perpendicular to virtual curve may be projected on the cutting plane.

In an embodiment, the cutting plane may be parallel to a coronal plane.

In an embodiment, the cutting plane may be input into a second artificial intelligence neural network, and the central separation data may be generated from the second artificial intelligence neural network.

In an embodiment, when the cutting plane is one or more, the second artificial intelligence neural network may be a two dimensional convolutional neural network.

In an embodiment, when the cutting plane is in a form of a stacked volume, the second artificial intelligence neural network may be a three dimensional convolutional neural network.

In an embodiment, the separating the end region of the inferior alveolar nerve canal may include generating an interest region based on the end feature point, and separating the end region of the inferior alveolar nerve canal in the interest region to generate end separation data.

In an embodiment, the interest region may be input into a third artificial intelligence neural network, and the end separation data may be generated from the third artificial intelligence neural network.

In an embodiment, the end region of the inferior alveolar nerve canal may be a region including an anterior loop, and the central region of the inferior alveolar nerve canal may be a region excluding the region including the anterior loop.

In an embodiment, the reconstructing the inferior alveolar nerve canal may include mapping central separation data, which are generated by separating the central region of the inferior alveolar nerve canal, onto the three dimensional volume data, and combining the mapped central separation data with end separation data, which are generated by separating the end region of the inferior alveolar nerve canal, to generate inferior alveolar nerve canal separation data.

In an embodiment, the inferior alveolar nerve canal separation data are refined and interpolated.

In an embodiment, the inferior alveolar nerve separation data are three dimensional volume data or mesh data.

In an embodiment, a program for executing the automated method for the inferior alveolar nerve canal segmentation of the three dimensional volume data on a computer may be stored in a computer readable medium.

According to the automated method for the inferior alveolar nerve canal segmentation of the three dimensional volume data, and the computer readable medium having the program, the central feature point and the end feature point may be determined from the three dimensional volume data including the inferior alveolar nerve canal. Based on the central feature point, the central region of the inferior alveolar nerve canal may be separated from the three dimensional volume data. Based on the end feature point, the end region of the inferior alveolar nerve canal may be separated from the three-dimensional volume data. The inferior alveolar nerve canal may be reconstructed based on the central region of the inferior alveolar nerve canal and the end region of the inferior alveolar nerve canal. Accordingly, a computational processing operation may require less time and be accurate.

In addition, at least one of determining the central feature point and the end feature point from the three dimensional volume data including the inferior alveolar nerve canal, separating the central region of the inferior alveolar nerve canal from the three dimensional volume data based on the central feature point, and separating the end region of the inferior alveolar nerve canal from the three dimensional volume data based on the end feature point may may be performed using an artificial intelligence neural network, such that the computational processing operation may require less time and be accurate.

### BRIEF DESCRIPTION OF THE DRAWINGS

The above and other features and advantages of the present inventive concept will become more apparent by describing in detailed embodiments thereof with reference to the accompanying drawings, in which:

FIG. 1 is a flowchart illustrating an automated method for an inferior alveolar nerve canal segmentation of three dimensional volume data according to an embodiment of the present inventive concept;

FIGS. 2 to 7 are conceptual diagrams illustrating feature points;

FIG. 8 is a flowchart illustrating separating a central region of an inferior alveolar nerve canal from three dimensional volume data based on a central feature point of FIG. 1;

FIGS. 9 to 14 are diagrams illustrating a feature point, a virtual curve, and a cutting plane;

FIGS. 15 and 16 are diagrams illustrating a cutting plane generated in various directions;

FIG. 17 is a diagram illustrating a second artificial intelligence neural network;

FIG. 18 is a flowchart illustrating separating an end region of an inferior alveolar nerve canal from a three dimensional volume data based on an end feature point of FIG. 1;

FIG. 19 is a diagram illustrating an interest region;

FIG. 20 is a diagram illustrating a third artificial intelligence neural network;

FIG. 21 is a flowchart illustrating reconstructing an inferior alveolar nerve canal based on a central region of the inferior alveolar nerve canal and an end region of the inferior alveolar nerve canal of FIG. 1; and

FIG. 22 is a diagram illustrating inferior alveolar nerve canal separation data of FIG. 21.

### DETAILED DESCRIPTION OF THE EMBODIMENTS

The present inventive concept now will be described more fully hereinafter with reference to the accompanying drawings, in which embodiments of the present inventive concept are shown. The present inventive concept may, however, be embodied in many different forms and should not be construed as limited to the embodiments set forth herein.

Rather, these embodiments are provided so that this disclosure will be thorough and complete, and will fully convey the scope of the present inventive concept to those skilled in the art. Like reference numerals refer to like elements throughout.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present inventive concept.

The terminology used herein is for the purpose of describing particular embodiments only and is not intended to be limiting of the present inventive concept. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the inventive concept as used herein.

Hereinafter, the present inventive concept will be explained in detail with reference to the accompanying drawings.

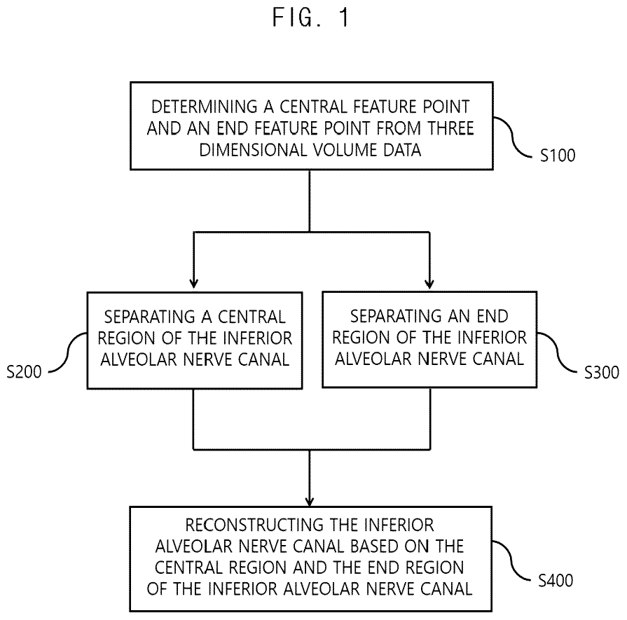

FIG. 1 is a flowchart illustrating an automated method for an inferior alveolar nerve canal segmentation of three dimensional volume data according to an embodiment of the present inventive concept.

Referring to FIG. 1, an automated method for an inferior alveolar nerve canal segmentation of three dimensional volume data according to an embodiment of the present inventive concept may include determining a central feature point and an end feature point from three dimensional volume data including an inferior alveolar nerve canal S100, separating a central region of the inferior alveolar nerve canal from the three dimensional volume data based on the central feature point S200, separating an end region of the inferior alveolar nerve canal from the three dimensional volume data based on the end feature point S300, and reconstructing the inferior alveolar nerve canal based on the central region of the inferior alveolar nerve canal and the end region of the inferior alveolar nerve canal S400.

In an embodiment, the end region of the inferior alveolar nerve canal may be a region including an anterior loop, and the central region of the inferior alveolar nerve canal may be a region excluding the region including the anterior loop. The central region of the inferior alveolar nerve canal may have a simple shape, while the end region of the inferior alveolar nerve canal may have a complex shape. Therefore, after the central region of the inferior alveolar nerve canal and the end region of the inferior alveolar nerve canal may be separated separately from the three dimensional volume data, the central region of the inferior alveolar nerve canal and the end region of the inferior alveolar nerve canal may be combined to reconstruct the inferior alveolar nerve canal.

The automated method for the inferior alveolar nerve canal segmentation of the three dimensional volume data may be performed by a computing device.

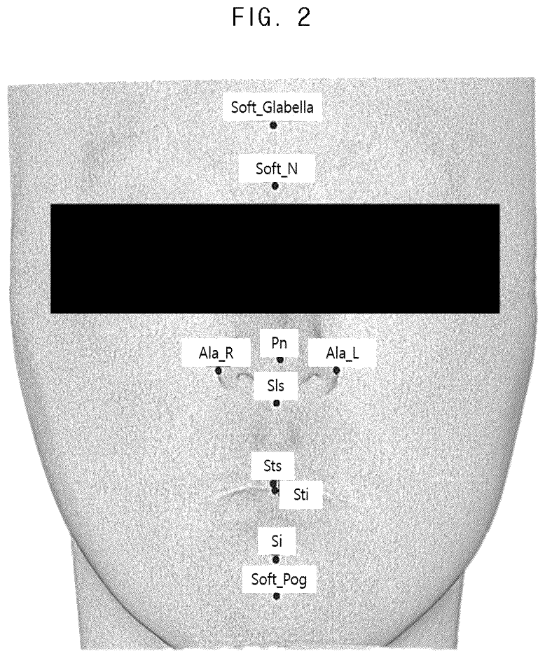

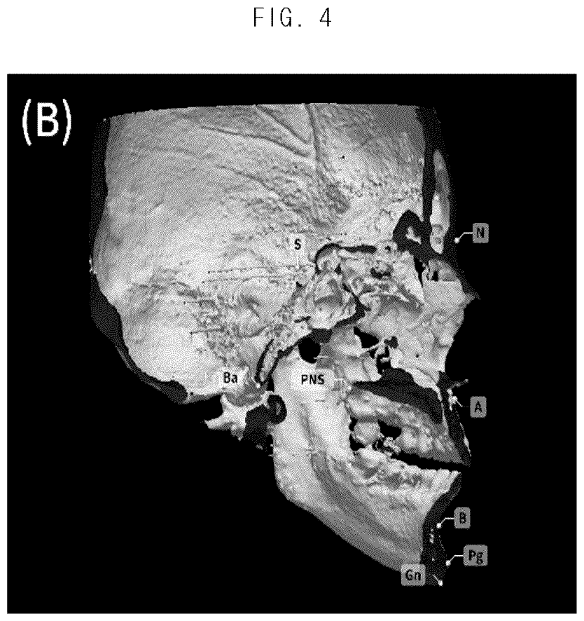

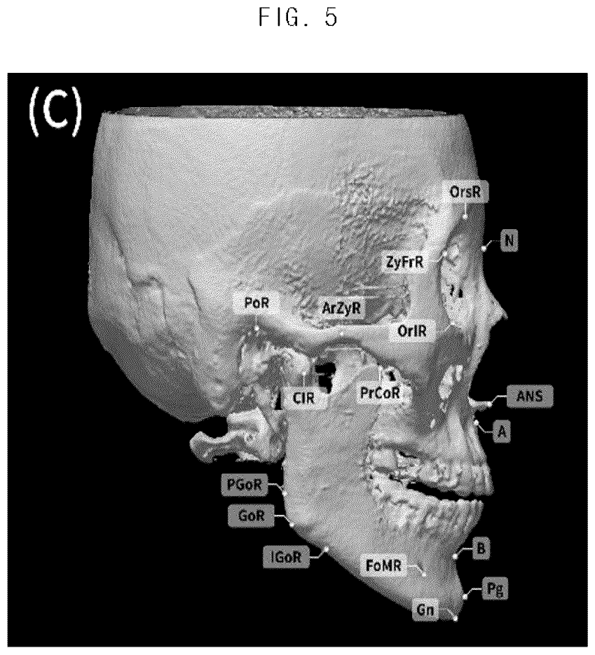

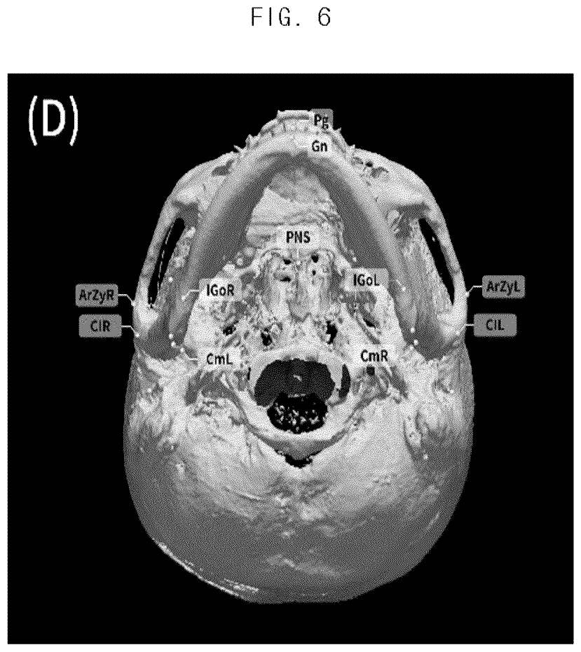

According to the automated method for the inferior alveolar nerve canal segmentation of the three dimensional volume data according to the present embodiment, the inferior alveolar nerve canal may be fully automatically segmented from the three dimensional volume data using deep learning. FIG. 1 describes an overall flowchart of the automated method for the inferior alveolar nerve canal segmentation of the three dimensional volume data. Each step is described in detail in FIGS. 2 to 22.

FIGS. 2 to 7 are conceptual diagrams illustrating feature points.

Referring to FIGS. 1 to 7, the automated method for the inferior alveolar nerve canal segmentation of the three dimensional volume data may include determining the central feature point and the end feature point from the three dimensional volume data including the inferior alveolar nerve canal S100.

Determining the central feature point and the end feature point from the three dimensional volume data including the inferior alveolar nerve canal S100 may be performed manually by a user or automatically by the deep learning. For example, determining the central feature point and the end feature point from the three dimensional volume data including the inferior alveolar nerve canal S100 may be performed using a first artificial intelligence neural network. That is, the three dimensional volume data may be input into the first artificial intelligence neural network, and the feature points may be output. The first artificial intelligence neural network is disclosed in Korean Patent Application No. 10-2373500, Korean Patent Application No. 10-2334480, etc., and a content disclosed in the patent registration documents may be incorporated into this specification as if fully disclosed.

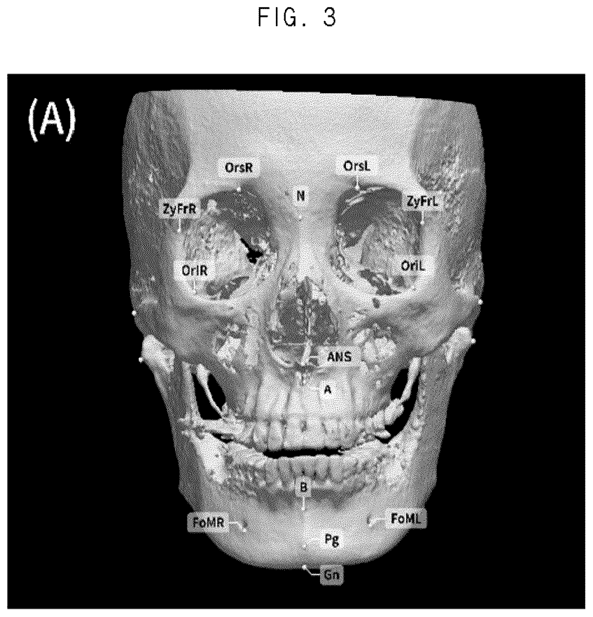

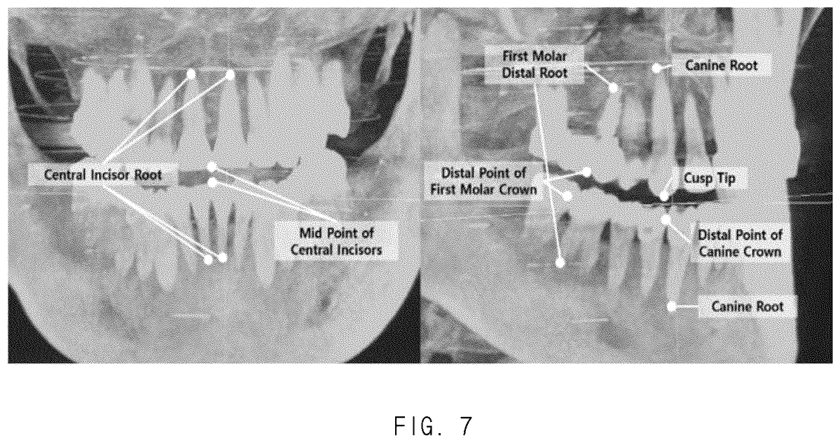

FIGS. 2 to 7 illustrate an example of the feature points. For example, the feature points may be maxillofacial feature points existing on a skin surface. FIG. 2 illustrates maxillofacial the feature points existing on the skin surface. Feature points of FIG. 2 may include Soft Tissue Glabella Soft_Glabella, Soft Tissue Nasion Soft_N, Pronasale Pn, Soft Tissue A-point Sls, Right Alar Base Ala_R, Left Alar Base Ala_L, Stomion Superior Sts, Stomion Inferius Sti, Mentolabial Sulcus Si, and Soft Tissue Pogonion Soft_Pog. For example, the feature points may be feature points associated with a maxillofacial bone. FIGS. 3 to 6 illustrate the feature points associated with the maxillofacial bone. Feature points of FISG. 3 to 6 may be expressed according to a frontal view A, a cutting plane of a side view B, a side view C, and a bottom view D. Feature points of FIGS. 3 to 6 are Sella S, Nasion N, Anterior Nasal Spine ANS, Point-A A, Posterior Nasal Spine PNS, Point-B B, Pogonion Pg, Gnathion Gn, Right/Left of Orbitale Superius OrSR/OrSL, Right/Left of Orbitale Inferius OriR/OriL, Right/Left of Sutura Zygomaticofrontale ZyFrR/ZyFrL, Right/Left of Foramen Mentale FoMR/FoML, Basion Ba, Right Porion POR, Right/Left of Condylus Medialis CmR/CmL, Right/Left of Condylus Lateralis CIR/CIL, Right/Left of Areus Zygomatieus ArZyR/ArZyL, Right/Left of Inferior Gonion IGOR/IGOL, Right/Left of Posterior Gonion PGoR/PGOL, Right of Processus Coronoideus PrCor, Right/Left of Gonion GoR/GoL. FIG. 7 illustrates feature points associated with teeth. Feature points of FIG. 7 may include Central Incisor Root, Mid Point of Central Incisors, First Molar Distal Root, Canine Root, Distal Point of First Molar Crown, Cusp Tip, Distal Point of Canine Crown, and Canine Root. However, feature points of the present inventive concept are not limited to feature points of FIGS. 2 to 7.

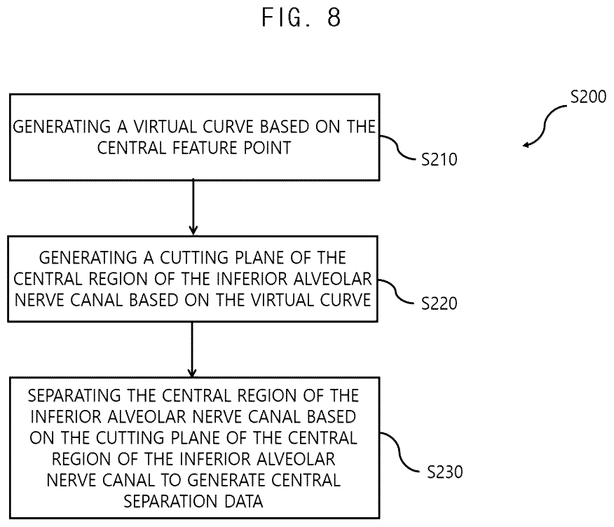

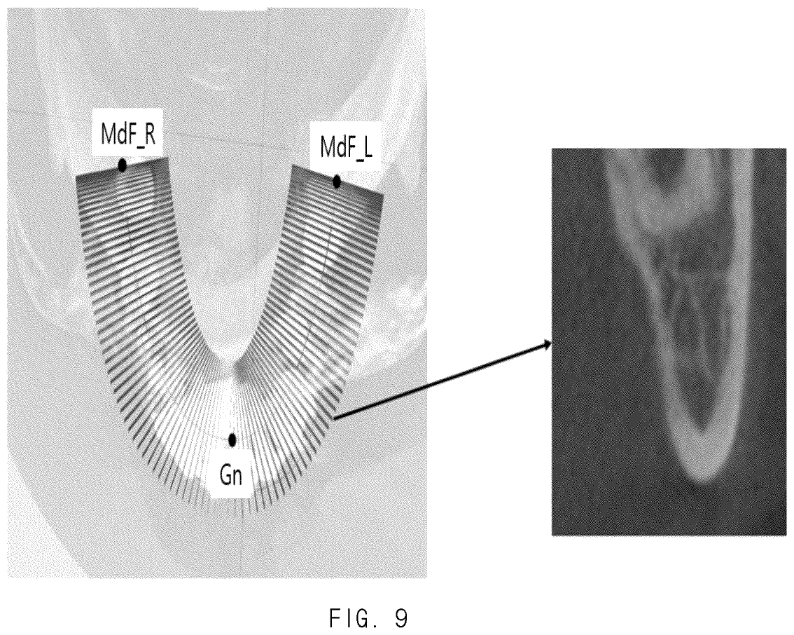

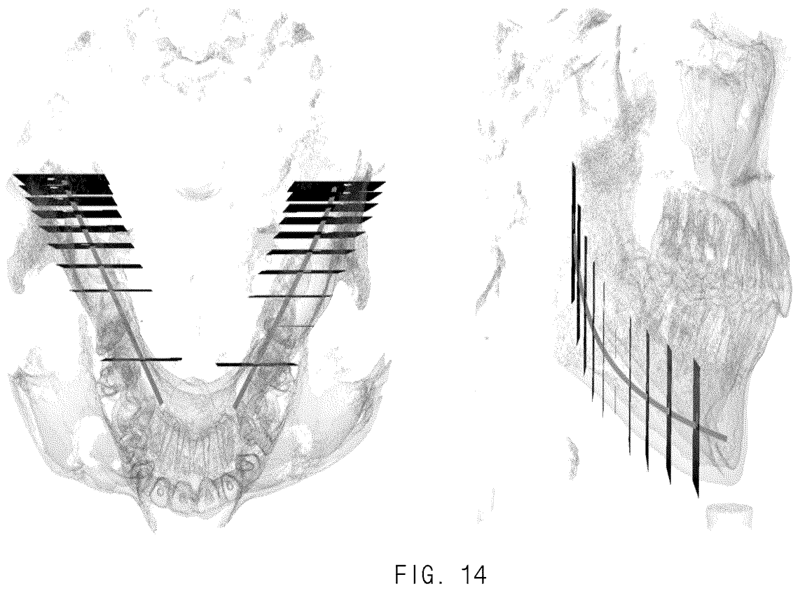

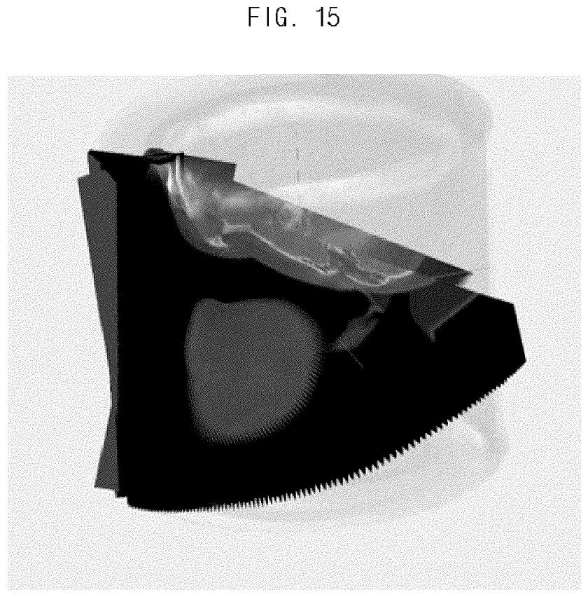

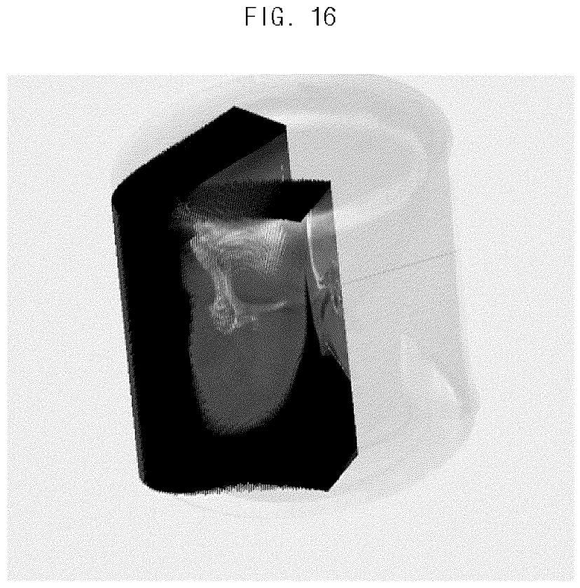

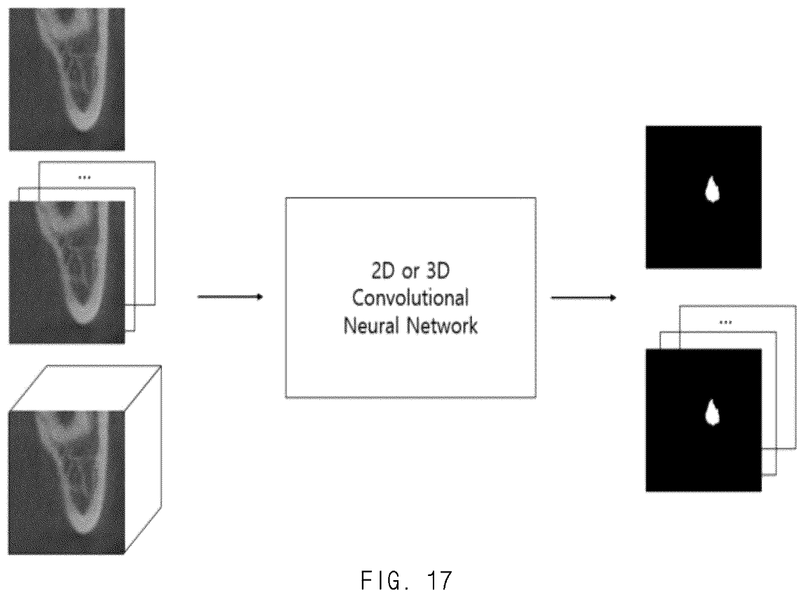

FIG. 8 is a flowchart illustrating separating a central region of an inferior alveolar nerve canal from three dimensional volume data based on a central feature point S200 of FIG. 1. FIGS. 9 to 14 are diagrams illustrating a feature point, a virtual curve, and a cutting plane. FIGS. 15 and 16 are diagrams illustrating a cutting plane generated in various directions. FIG. 17 is a diagram illustrating a second artificial intelligence neural network.

Referring to FIGS. 1 to 17, the automated method for the inferior alveolar nerve canal segmentation of the three dimensional volume data may include separating the central region of the inferior alveolar nerve canal from the three dimensional volume data based on the central feature point S200.

Separating the central region of the inferior alveolar nerve canal from the three dimensional volume data based on the central feature point S200 may include generating a virtual curve based on the central feature point S210, generating a cutting plane of the central region of the inferior alveolar nerve canal based on the virtual curve S220, and separating the central region of the inferior alveolar nerve canal based on the cutting plane of the central region of the inferior alveolar nerve canal to generate central separation data S230.

The virtual curve may be generated based on the central feature point. The central feature point may include at least three feature points. In an embodiment, the virtual curve may be a spline curve which directly passes through the central feature point. In an embodiment, the virtual curve may be a Bezier curve (Bézier Curve which does not directly pass through the central feature point. The Bezier curve is a curve which uses a linear interpolation. In addition, the cutting plane may be generated based on the virtual curve. However, the present inventive concept is not limited thereto. The virtual curve may be any curve other than the spline curve and the Bezier curve.

For example, as shown in FIG. 9, the central feature point may be Right Mandibular Foramen MdF_R, Gnathion Gn, and Left Mental Foramen MdF_L. A virtual curve of FIG. 9 may be a spline curve which passes through a central feature point of FIG. 9. A cutting plane of FIG. 9 may be generated based on a virtual curve of FIG. 9. A right diagram of FIG. 9 illustrates an example of a cutting plane of FIG. 9.

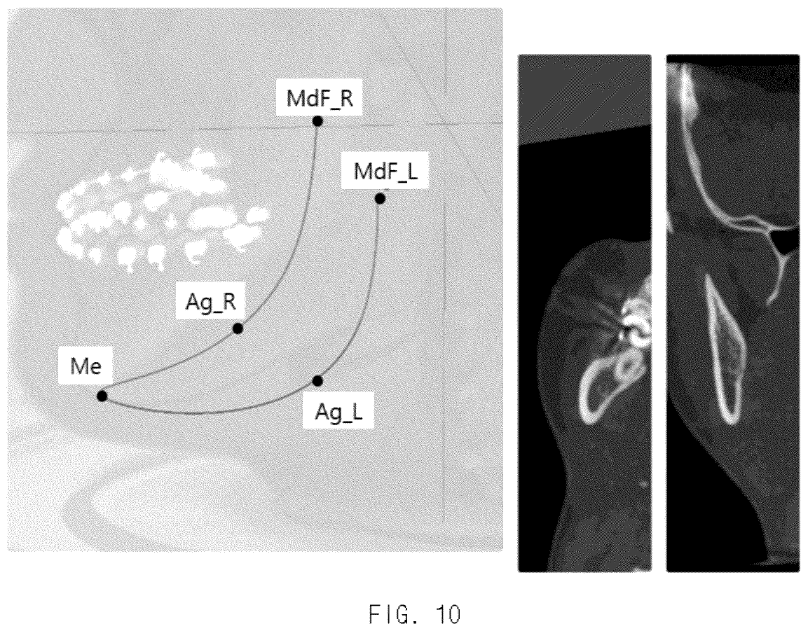

For example, as shown in FIG. 10, the central feature point may be Right Mandibular Foramen MdF_R, Right Antegonion Ag_R, Menton Me, Left Antegonion Ag_L, and Left Mental Foramen MdF_L. A virtual curve of FIG. 10 may be a spline curve which passes through a central feature point of FIG. 10. A right diagram of FIG. 10 illustrates an example of a cutting plane of FIG. 10.

For example, as shown in FIG. 11, the central feature point may be Right Porian Po_R, Right Mandibular Foramen MdF_R, Menton Me, Left Mandibular Foramen MdF_L, and Left Porian Po_L. A virtual curve of FIG. 11 may be a spline curve which passes through a central feature point of FIG. 11. A right diagram of FIG. 11 illustrates an example of a cutting plane of FIG. 11.

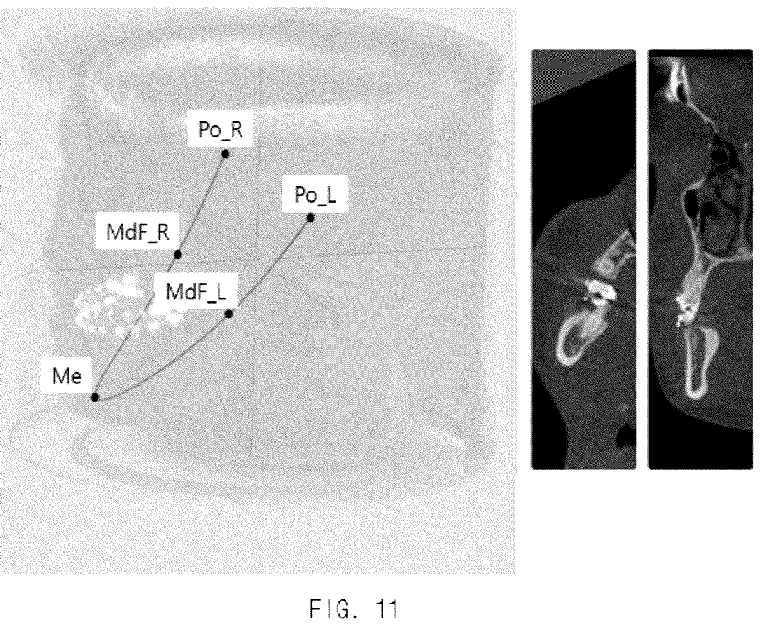

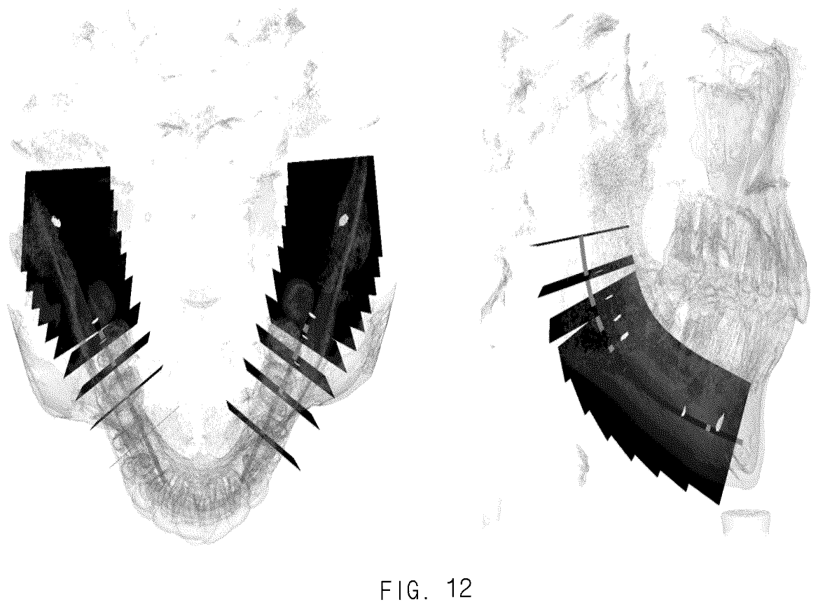

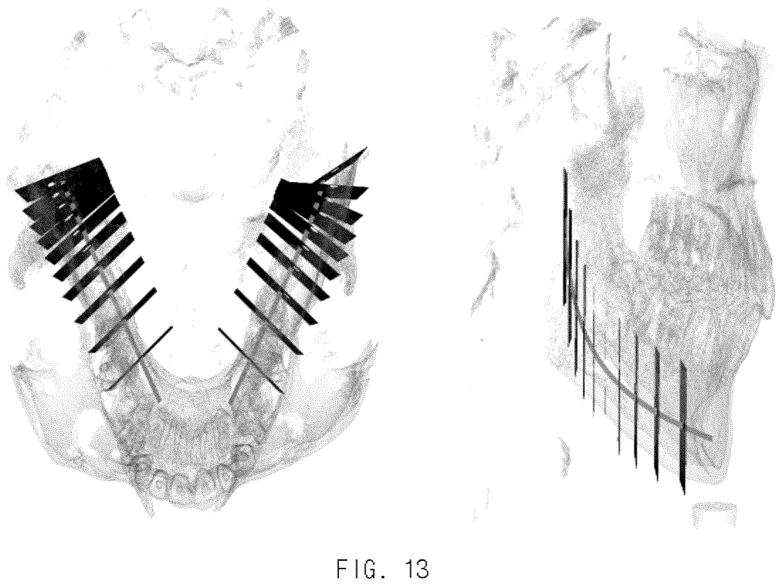

The cutting plane may be generated in various directions based on the virtual curve. As shown in FIG. 12, in an embodiment, the cutting plane may be perpendicular to the virtual curve. As shown in FIG. 13, in an embodiment, an intermediate cutting plane perpendicular to the virtual curve may be a projected plane. As shown in FIG. 14, in an embodiment, the cutting plane may be parallel to a coronal plane.

For example, FIG. 15 shows an example of the cutting plane perpendicular to the virtual curve. For example, FIG. 16 shows an example of a projected plane of the intermediate cutting plane perpendicular to the virtual curve.

The coronal plane is one of planes representing a human body. For example, the planes representing the human body may include a sagittal plane, the coronal plane, and a transverse plane. The sagittal plane divides the human body into a left side and a right side. The coronal plane divides the human body into a front side and a back side. The transverse plane divides the human body into an upper side and a lower side.

Separating the central region of the inferior alveolar nerve canal based on the cutting plane of the central region of the inferior alveolar nerve canal to generate the central separation data S230 may be performed using a second artificial intelligence neural network. That is, the cutting plane may be input to the second artificial intelligence neural network, and the central separation data may be output. The second artificial intelligence neural network may be a Convolutional Neural Network (CNN).

In an embodiment, the second artificial intelligence neural network may be a two dimensional convolutional neural network. The cutting plane may be one or more. In this case, the cutting plane may be a two dimensional image. Therefore, the two dimensional convolutional neural network may be used.

In an embodiment, the second artificial intelligence neural network may be a three dimensional convolutional neural network. The cutting plane may be in a form of a stacked volume. In this case, the cutting plane may be a three dimensional image. Therefore, the three dimensional convolutional neural network may be used.

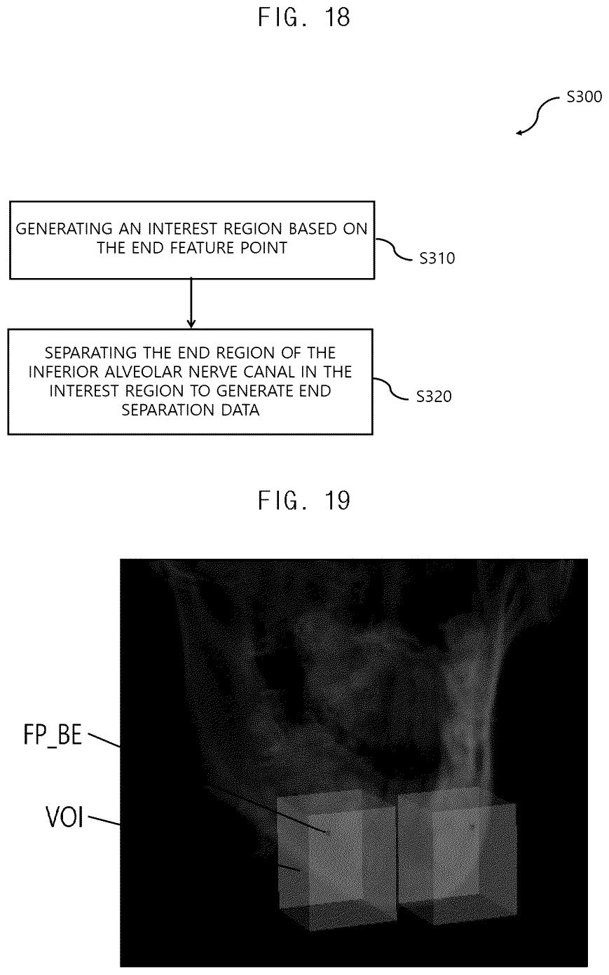

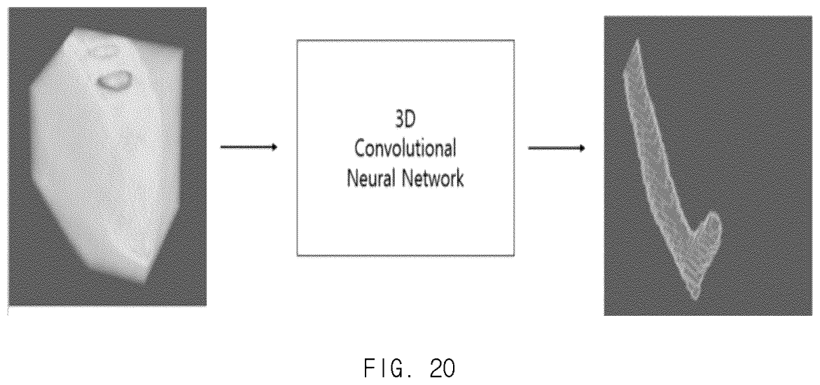

FIG. 18 is a flowchart illustrating separating an end region of an inferior alveolar nerve canal from a three dimensional volume data based on an end feature point S300 of FIG. 1. FIG. 19 is a diagram illustrating an interest region VOI. FIG. 20 is a diagram illustrating a third artificial intelligence neural network.

Referring to FIGS. 1 to 20, the automated method for the inferior alveolar nerve canal segmentation of the three dimensional volume data may include separating the end region of the inferior alveolar nerve canal from the three dimensional volume data based on the end feature point FP_BE S300.

Separating the end region of the inferior alveolar nerve canal from the three dimensional volume data based on the end feature point FP_BE S300 may include generating an interest region VOI based on the end feature point FP_BE S310, and separating the end region of the inferior alveolar nerve canal in the interest region VOI to generate end separation data S320.

The interest region VOI may be generated based on the end feature point FP_BE. Specifically, the end feature point FP_BE may be a point located around the anterior loop. Therefore, the interest region VOI may be a region including the anterior loop. In FIG. 19, the end feature point FP_BE is shown as a dot, and the interest region VOI is shown as a rectangular parallelepiped (or a cube) including the dot.

Separating the end region of the inferior alveolar nerve canal in the interest region VOI to generate end separation data S320 may be performed using a third artificial intelligence neural network. That is, the interest region VOI may be input to the third artificial intelligence neural network, and the end separation data may be output. The third artificial intelligence neural network may be the convolutional neural network.

In an embodiment, the third artificial intelligence neural network may be a three dimensional convolutional neural network. The interest region VOI may be a three dimensional image. Therefore, the three dimensional convolutional neural network may be used.

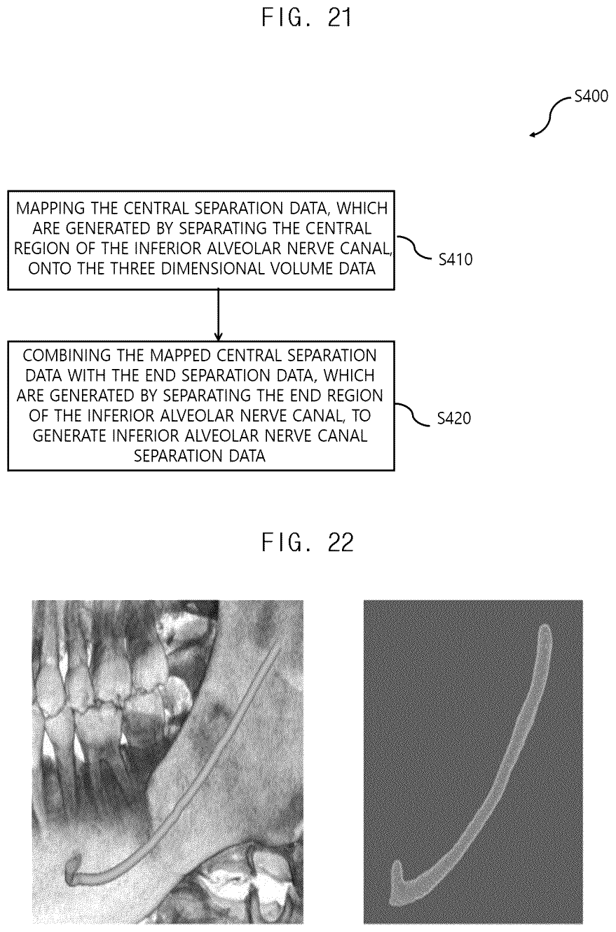

FIG. 21 is a flowchart illustrating reconstructing an inferior alveolar nerve canal based on a central region of the inferior alveolar nerve canal and an end region of the inferior alveolar nerve canal S400 of FIG. 1. FIG. 22 is a diagram illustrating inferior alveolar nerve canal separation data of FIG. 21.

Referring to FIGS. 1 to 22, the automated method for the inferior alveolar nerve canal segmentation of the three dimensional volume data may include reconstructing the inferior alveolar nerve canal based on the central region of the inferior alveolar nerve canal and the end region of the inferior alveolar nerve canal S400

Reconstructing the inferior alveolar nerve canal based on the central region of the inferior alveolar nerve canal and the end region of the inferior alveolar nerve canal S400 may include mapping the central separation data, which is generated by separating the central region of the inferior alveolar nerve canal, onto the three dimensional volume data S410, and combining the mapped central separation data with the end separation data, which is generated by separating the end region of the inferior alveolar nerve canal, to generate inferior alveolar nerve canal separation data S420.

As described in FIGS. 8 to 17, the central separation data may be generated. The central separation data may be mapped to the three dimensional volume data. As described in FIGS. 18 to 20, the end separation data may be generated. As shown in a left diagram of FIG. 22, the inferior alveolar nerve canal separation data may be generated by combining the mapped central separation data and the end separation data. The inferior alveolar nerve canal separation data included in the left diagram of FIG. 22 may be converted into mesh data using a method such as a marching cube. In an embodiment, the inferior alveolar nerve canal separation data may be the three dimensional volume data or the mesh data. The mesh data may include three dimensional points (Vertices) and a triangular face (Triangle) or a rectangular face (Rectangle) created by connecting the points. However, the present inventive concept is not limited thereto. The mesh data may be any polygon other than the triangular face and the rectangular face.

The inferior alveolar nerve canal separation data may be refined and interpolated. In particular, the interpolation may be performed on the central separation data or between the central separation data and the end separation data.

According to the present embodiment, the central feature point and the end feature point FP_BE may be determined from the three dimensional volume data including the inferior alveolar nerve canal. Based on the central feature point, the central region of the inferior alveolar nerve canal may be separated from the three dimensional volume data. Based on the end feature point FP_BE, the end region of the inferior alveolar nerve canal may be separated from the three dimensional volume data. The inferior alveolar nerve canal may be reconstructed based on the central region of the inferior alveolar nerve canal and the end region of the inferior alveolar nerve canal. Accordingly, a computational processing operation may require less time and be accurate.

In addition, at least one of determining the central feature point and the end feature point FP_BE from the three dimensional volume data including the inferior alveolar nerve canal S100, separating the central region of the inferior alveolar nerve canal from the three dimensional volume data based on the central feature point S200, and separating the end region of the inferior alveolar nerve canal from the three dimensional volume data based on the end feature point S300 may may be performed using an artificial intelligence neural network, such that the computational processing operation may require less time and be accurate.

According to an embodiment of the present inventive concept, a non-transitory computer-readable storage medium having stored thereon program instructions of the automated method for the inferior alveolar nerve canal segmentation of the three dimensional volume data according to an embodiment of the present inventive concept may be provided. The above mentioned method may be written as a program executed on the computer. The method may be implemented in a general purpose digital computer which operates the program using a computer-readable medium. In addition, the structure of the data used in the above mentioned method may be written on a computer readable medium through various means. The computer readable medium may include program instructions, data files and data structures alone or in combination. The program instructions written on the medium may be specially designed and configured for the present inventive concept, or may be generally known to a person skilled in the computer software field. For example, the computer readable medium may include a magnetic medium such as a hard disk, a floppy disk and a magnetic tape, an optical recording medium such as CD-ROM and DVD, a magneto-optical medium such as floptic disc and a hardware device specially configured to store and execute the program instructions such as ROM, RAM and a flash memory. For example, the program instructions may include a machine language codes produced by a compiler and high-level language codes which may be executed by a computer using an interpreter or the like. The hardware device may be configured to operate as one or more software modules to perform the operations of the present inventive concept.

In addition, the above mentioned automated method for the inferior alveolar nerve canal segmentation of the three dimensional volume data may be implemented in a form of a computer-executed computer program or an application which are stored in a storage method.

The present inventive concept relates to the automated method for the inferior alveolar nerve canal segmentation of the three dimensional volume data, and a computer-readable medium having the program for executing the same, which may reduce an effort and a time for a computational processing operation and improve an accuracy and an productivity.

The foregoing is illustrative of the present inventive concept and is not to be construed as limiting thereof. Although a few embodiments of the present inventive concept have been described, those skilled in the art will readily appreciate that many modifications are possible in the embodiments without materially departing from the novel teachings and advantages of the present inventive concept. Accordingly, all such modifications are intended to be included within the scope of the present inventive concept as defined in the claims. In the claims, means-plus-function clauses are intended to cover the structures described herein as performing the recited function and not only structural equivalents but also equivalent structures. Therefore, it is to be understood that the foregoing is illustrative of the present inventive concept and is not to be construed as limited to the specific embodiments disclosed, and that modifications to the disclosed embodiments, as well as other embodiments, are intended to be included within the scope of the appended claims. The present inventive concept is defined by the following claims, with equivalents of the claims to be included therein.

What is claimed is:

1. An automated method for an inferior alveolar nerve canal segmentation of three dimensional volume data, the method comprising:
determining a central feature point and an end feature point from the three dimensional volume data including an inferior alveolar nerve canal;
separating a central region of the inferior alveolar nerve canal from the three dimensional volume data based on the central feature point;
separating an end region of the inferior alveolar nerve canal from the three dimensional volume data based on the end feature point; and
reconstructing the inferior alveolar nerve canal based on the central region of the inferior alveolar nerve canal and the end region of the inferior alveolar nerve canal.

2. The automated method of claim 1, wherein the three dimensional volume data are input into a first artificial intelligence neural network, and
wherein the central feature point and the end feature point are determined from the first artificial intelligence neural network.

3. The automated method of claim 1, wherein the central feature point includes at least three feature points.

4. The automated method of claim 1, wherein the end feature point includes at least two feature points.

5. The automated method of claim 1, wherein the separating the central region of the inferior alveolar nerve canal includes:
generating a virtual curve based on the central feature point;
generating a cutting plane of the central region of the inferior alveolar nerve canal based on the virtual curve; and
separating the central region of the inferior alveolar nerve canal based on the cutting plane of the central region of the inferior alveolar nerve canal to generate central separation data.

6. The automated method of claim 5, wherein the virtual curve is a spline curve which directly passes through the central feature point.

7. The automated method of claim 5, wherein the virtual curve is a Bezier curve which does not directly pass through the central feature point.

8. The automated method of claim 5, wherein the cutting plane is perpendicular to the virtual curve.

9. The automated method of claim 5, wherein an intermediate cutting plane perpendicular to virtual curve is projected on the cutting plane.

10. The automated method of claim 5, wherein the cutting plane is parallel to a coronal plane.

11. The automated method of claim 5, wherein the cutting plane is input into a second artificial intelligence neural network, and
wherein the central separation data are generated from the second artificial intelligence neural network.

12. The automated method of claim 11, wherein, when the cutting plane is one or more, the second artificial intelligence neural network is a two dimensional convolutional neural network.

13. The automated method of claim 11, wherein, when the cutting plane is in a form of a stacked volume, the second artificial intelligence neural network is a three dimensional convolutional neural network.

14. The automated method of claim 1, wherein the separating the end region of the inferior alveolar nerve canal includes:
generating an interest region based on the end feature point; and
separating the end region of the inferior alveolar nerve canal in the interest region to generate end separation data.

15. The automated method of claim 14, wherein the interest region is input into a third artificial intelligence neural network, and
wherein the end separation data are generated from the third artificial intelligence neural network.

16. The automated method of claim 1, wherein the end region of the inferior alveolar nerve canal is a region including an anterior loop, and
wherein the central region of the inferior alveolar nerve canal is a region excluding the region including the anterior loop.

17. The automated method of claim 1, wherein the reconstructing the inferior alveolar nerve canal includes:
mapping central separation data, which are generated by separating the central region of the inferior alveolar nerve canal, onto the three dimensional volume data; and
combining the mapped central separation data with end separation data, which are generated by separating the end region of the inferior alveolar nerve canal, to generate inferior alveolar nerve canal separation data.

18. The automated method of claim 17, wherein the inferior alveolar nerve canal separation data are refined and interpolated.

19. The automated method of claim 17, wherein the inferior alveolar nerve separation data are three dimensional volume data or mesh data.

20. A non-transitory computer-readable storage medium having stored thereon program instructions, the program instructions executable by at least one hardware processor to:
determining a central feature point and an end feature point from three dimensional volume data including an inferior alveolar nerve canal;
separating a central region of the inferior alveolar nerve canal from the three dimensional volume data based on the central feature point;
separating an end region of the inferior alveolar nerve canal from the three dimensional volume data based on the end feature point; and
reconstructing the inferior alveolar nerve canal based on the central region of the inferior alveolar nerve canal and the end region of the inferior alveolar nerve canal.
